(19) United States
(12) Patent Application Publication
(10) Pub. No.: US 2010/0234832 A1
(43) Pub. Date: Sep. 16, 2010

(54) METHOD FOR VASCULAR TREATMENT

(76) Inventors: Julio H. G. Ferreira, Porto Alegre (BR); Antonio Carlos Reichelt, Porto Alegre (BR)

Correspondence Address: BOLESH J. SKUTNIK PhD, JD, 515 Shaker Road, East Longmeadow, MA, 01028, US

(21) Appl. No.: 12/721,925

(22) Filed: Mar. 11, 2010

Related U.S. Application Data
(60) Provisional application No. 61/160,212, filed on Mar. 13, 2009.

(51) Int. Cl. A61B 18/24

(52) U.S. Cl. 606/2

(57) ABSTRACT

A method is disclosed for improved minimally invasive vascular treatment. The procedure consists in performing a femoral nerve block, in order to achieve a sensorial block but not a motor block in the lower extremity. A femoral nerve block carries low risk of complications and when combined with block of the sciatic nerve provides anesthesia of almost the entire lower extremity. In a preferred embodiment, 20 ml of about 0.1% to about 0.5% lidocaine are injected under echographic/ultrasound control. After the anesthesia takes effect, a needle or other means is inserted through the skin into the vein to be treated and an optical fiber is introduced inside it. In a preferred embodiment, optical fiber is a radial emitting fiber and wavelength used is 1470 nm. Laser radiation is emitted while withdrawing the optical fiber, choosing appropriate irradiation parameters for each case. Nerve block technique disclosed can be used for vascular treatment in general, particularly whenever local energy techniques are employed, whether they be laser, thermal or RF sources, and anywhere sensorial anesthesia is needed and it is desirable for the patient to retain motor control. This new vascular procedure facilitates physician tasks since it is a very simple method to perform. Real time ultrasound monitoring of vein closure process is feasible, allowing the surgeon to use the minimum amount of energy possible, thus minimizing damage to surrounding tissues. This, in turn, considerably diminishes possibility of human error and as a consequence enhances patient safety. Furthermore, patient comfort is enhanced due to an excellent analgesia, during and after the treatment.

### DOMESTIC PRIORITY UNDER 35 USC 119(E)

This application claims the benefit and priority of U.S. Provisional Application Ser. No. 61/160,212 filed Mar. 13, 2009, entitled “Method for Vascular Treatment” by Julio H. G. Ferreira and Antonio Carlos Reichelt, which is incorporated by reference herein.

### BACKGROUND OF THE INVENTION

1. Field of the Invention

The present invention relates to endovascular treatments and in particular, to the treatment of vascular pathologies, such as venous insufficiency, with energy emitting devices using an optical fiber set and low power densities.

2. Invention Disclosure Statement

The human venous system of the lower limbs consists essentially of the superficial venous system and the deep venous system, both connected by perforating veins. The superficial system comprises the great and the small saphenous veins, while the deep venous system includes the anterior and posterior tibial veins, which converge to form the popliteal vein near the knee. The popliteal vein, in turn, becomes the femoral vein when joined by the small saphenous vein.

The venous system comprises valves, whose main function is to achieve unidirectional blood flow back to the heart. Venous valves are usually bicuspid valves, with each cusp forming a blood reservoir, which force their free surfaces together under retrograde blood pressure. As a consequence, when properly operating, retrograde blood flow is prevented, allowing only antegrade flow to the heart. A valve becomes incompetent when their cusps are unable to seal properly under retrograde pressure gradient, so retrograde blood flow occurs. When retrograde blood flow occurs, pressure increases in the lower venous sections, dilating veins and usually leading to additional valvular failure.

Valvular failure, usually referred to as venous insufficiency, is a chronic disease that can lead to skin discoloration, varicose veins, pain, swelling and ulcerations. Varicose veins refer to blood vessels that have become enlarged and twisted and have progressively lost their wall elasticity. Owing to the widening of the blood vessels, vein valves cannot be completely closed and veins lose their ability to carry blood back to the heart. This leads to an accumulation of blood inside the vessels, enlarging and twisting the veins even more. Furthermore, varicose veins usually have a blue or purple color and may protrude twisted above the surface of the skin, being responsible of their characteristically unattractive appearance. They are commonly formed in the superficial veins of the legs, which are subject to high pressure when standing. Other types of varicose veins include venous lakes, reticular veins and telangiectasias.

There are a number of treatments available intending to eradicate these kinds of vascular pathologies. Some of them only consist in relief of symptoms because they do not prevent new varicose veins from forming. These include elevating the legs by lying down or using a footstool when sitting, elastic stockings and exercise.

Varicose veins are frequently treated by eliminating the insufficient veins. This forces the blood to flow through the remaining healthy veins. Various methods can be used to eliminate the problem insufficient veins, including, sclerotherapy, surgery, electro-cautery, and laser treatments.

Sclerotherapy uses a fine needle to inject a solution directly into the vein. This solution irritates the lining of the vein, causing it to swell and the blood to clot. The vein turns into scar tissue that fades from view. Some physicians treat both varicose and spider veins with sclerotherapy. Today, the substances most commonly used are hypertonic saline, dextrose, polidocanol, glycerin, ethanolamine oleate or sotradecol (sodium tetradecyl sulfate). The sclerosant acts upon the inner lining of the vein walls causing them to occlude and block blood flow. This method has numerous complications. People with allergies may suffer allergic reactions, occasionally severe. The sclerosant may burn the skin, if the needle is not properly inserted or permanently mark or stain the skin. Furthermore, sclerotherapy can lead occasionally to blood clots. Moreover, treatment is limited to veins of a particular size and range as larger varicose veins may be more likely, according to many studies, to recur if treated with sclerotherapy.

Surgery to treat varicose veins, commonly referred to as “vein stripping”, is usually done under general, local or partial anesthesia. Here, the problematic veins are stripped out by passing a flexible device through the vein and removing it through an incision near the groin. Smaller tributaries of these veins also are stripped with this device or removed through a series of small incisions. Those veins that connect to the deeper veins are then tied off. Surgery also results in scarring where small incisions are made and may occasionally cause blood clots. Furthermore, procedures are long and require long recovery periods.

In addition, these surgical therapies show several disadvantages compared to other approaches. One of them is the need of general, loco-regional or peridural anesthesia. Furthermore, these procedures may damage collateral branches of the vein which may consequently bleed, giving rise to hematomas, or may lead to other complications such as blood loss, pain, infection, nerve injury and swelling. Moreover, because of the damage done to the treated area, patients may have pain and discomfort for hours and days after surgery. Side effects of this method of removing varicose veins are those for any surgery performed under anesthesia, including nausea, vomiting, and the risk of wound infection.

Another well known method of treating insufficient veins is through the use of radio frequency (RF). An example of the application of this method can be found in U.S. Patent Publication No 2006/0069471, by Farley et al. Electrodes are introduced through a catheter inside the vein to be treated for insufficiency and RF is applied to cause selective heating of the vein. The catheter is positioned within the vein to be treated and the electrodes on the catheter are moved toward one side of the vein. RF energy is applied in a directional manner from electrodes at the working end of a catheter to cause localized heating and corresponding shrinkage of the adjacent venous tissue. This method has some disadvantages. The use of RF energy in the treatment of vein insufficiency may be ineffective in certain cases and interventions can be lengthy and stressful for the patient. Nevertheless, it is still a popular local energy treatment among physicians.

There are minimally invasive surgical options for the treatment of blood vessels. The alternatives preferred by those skilled in the art are those that use laser radiation. Laser surgery has been improved due to new diode laser systems. For endovascular laser surgery, laser radiation applies thermal energy to the vein with the aid of an optical fiber, and while it is withdrawn, the vein closes and eventually disappears through absorption. In these and other cases, endovascular laser treatment provides an effective technique for eliminating skin and vascular problems. Moreover, it still provides a closer access to the treatment area, allowing less powerful laser equipments and less skin or healthy tissue damage. This in turn reduces costs while at the same time provides a shorter recovery period due to less skin and surrounding healthy tissue damage.

An important factor determining surgery outcome and patient satisfaction is the level of discomfort and pain suffered by the patient. Different variables can determine level of pain, such as the technique chosen, equipment used, surgeon skill and even the patient's subjective capacity to withstand pain.

For example, endovenous laser treatment with a fiber emitting radially at 1470 nm can be substantially painless. U.S. Provisional Patent Application No. 61/079,024 by Neuberger teaches a device and method for this type of surgery. Other techniques can cause different degrees of discomfort and pain to the patient.

Anesthetic techniques commonly used in surgical treatment of insufficient veins, include general anesthesia, central nerve block and tumescent anesthesia.

General anesthesia and central nerve block have many possible adverse effects. They can cause muscle rigidity and airway irritation at induction, hypotension and bradycardia intraoperatively, and nausea and vomiting after surgery. If used in day surgery the time to discharge is often after several hours.

Tumescent anesthesia is a technique for delivery of local anesthesia that improves safety by using pharmacokinetic principles to achieve extensive regional anesthesia of skin and subcutaneous tissue. The subcutaneous infiltration of a large volume of tumescent liquid causes the targeted tissue to become swollen and firm, or tumescent, and permits procedures to be performed on patients without subjecting them to the inherent risks of general/central anesthesia and blood loss. Tumescent anesthesia offers improved hemostasis, reduced hematoma and hyperpigmentation, pre-dissection of the vein from surrounding tissue by the solution pumped into the subcutaneous space, cooling effect during endovenous ablation of varicose veins. This technique is preferred by many authors. However, success of tumescent anesthesia depends largely on surgeon expertise and precision of the injections. Also, the injection of large volumes of solution may be at times inconvenient. Furthermore, Holdstock et al. (J. M. Holdstock, P. Marsh, M. S. Whiteley, B. A. Price. It is Possible to Cause Damage to a Laser Fibre during Delivery of Tumescent Anaesthesia fir Endovenous Laser Ablation (EVLA), Eur J Vasc Endovasc Surg (2008) 36, 473-476) suggest that in some cases, the technique of tumescent anesthesia may cause damage to laser fiber.

Femoral nerve block provides much of what can be achieved by tumescent anesthesia and avoids injection of large volumes of solution. However, tumescent anesthesia technique has historically been considered to be necessary for local energy intravenous treatments, such as RF or laser to provide the necessary heat sink and protection of nerves surrounding veins, while local anesthetics have been limited to physical techniques such as vein stripping.

There is therefore a need for a new method to address prior art disadvantages. This invention addresses this need.

### OBJECTIVES AND BRIEF SUMMARY OF THE INVENTION

It is an objective of the present invention to provide a method of anesthetic use for improved vascular treatment of venous insufficiency.

It is also an objective of the present invention to provide a method for safer, painless and more reliable vascular treatment to accomplish an effective insufficient vein closure.

It is yet another objective of the present invention to provide an endovascular radiation method that minimizes pain during and after the procedure.

It is another objective of the present invention to treat insufficient veins without introducing complications arising from administration of large amounts of anesthesia around them.

It is yet another objective of the present invention to provide a simpler method to that minimizes the possibility of harming patient or damaging fiber due to procedural errors by the physician.

Briefly stated, a method is disclosed for improved minimally invasive vascular treatment. The procedure consists in performing a femoral nerve block, in order to achieve a sensorial block but not a motor block in the lower extremity. A femoral nerve block carries low risk of complications and when combined with block of the sciatic nerve provides anesthesia of almost the entire lower extremity. In a preferred embodiment, 20 ml of about 0.1% to about 0.5% lidocaine are injected under echographic/ultrasound control. After the anesthesia takes effect, a needle or other means is inserted through the skin into the vein to be treated and an optical fiber is introduced inside it. In a preferred embodiment, optical fiber is a radial emitting fiber and wavelength used is 1470 nm. Laser radiation is emitted while withdrawing the optical fiber, choosing appropriate irradiation parameters for each case. Nerve block technique disclosed can be used for vascular treatment in general, particularly whenever local energy techniques are employed, whether e.g. they be laser, thermal or RE sources, and anywhere sensorial anesthesia is needed and it is desirable for the patient to retain motor control. This new vascular procedure facilitates physician tasks since it is a very simple method to perform. Real time ultrasound monitoring of vein closure is (thus) possible, allowing the surgeon to use a minimum amount of energy, thus avoiding damage to surrounding tissues. This, in turn, considerably diminishes possibility of human error and as a consequence enhances patient safety. Furthermore, patient comfort is enhanced due to an excellent analgesia, during and after the treatment.

The above and other objects, features and advantages of the present invention will become apparent from the following description read in conjunction with the accompanying drawings (in which like reference numbers in different drawings designate the same elements).

### BRIEF DESCRIPTION OF FIGURES

FIG. 1 depicts a preferred embodiment of present invention describing the main steps of the disclosed procedure.

FIG. 2 depicts another preferred embodiment of present invention describing the main steps of a procedure including a local energy source.

### DETAILED DESCRIPTION OF PREFERRED EMBODIMENTS

As previously mentioned, prior art vein treatments present numerous drawbacks. Sclerotherapy, for instance, may cause allergic reactions, skin burnt and stains, and blood clots. In addition, treatment is limited to veins of a particular size and range as larger varicose veins may be more likely, according to many studies, to recur if treated with sclerotherapy. Vein stripping shows several disadvantages compared to other approaches: general, loco-regional or peridural anesthesia necessity (with their associated side effects); it often damages collateral branches of the vein with consequent bleeding, hematomas, or other complications such as blood loss, pain, infection, nerve injury and swelling. This invasive procedure involves damage to the treated area, leading to patient pain and discomfort for hours and even days after surgery. Finally, the use of RF energy in the treatment of vein insufficiency may be ineffective in certain cases and interventions can be lengthy and stressful for the patient. Nevertheless, it is a popular local energy treatment among physicians.

Laser procedures, minimally invasive treatment of blood vessels, overcome the majority of these disadvantages. They provide a closer access to the treatment area, allowing less powerful laser equipments and less damage to skin or to healthy tissue. This in turn reduces costs while at the same time provides a shorter recovery period due to less damage to skin and to surrounding healthy tissue. Laser procedures are safe, with low vein perforation risks, fast and easy to perform for those skilled in the art.

Regarding anesthesia, main drawbacks of commonly used techniques in prior art can be summarized. General anesthesia and central nerve block can cause muscle rigidity and airway irritation at induction, hypotension and bradycardia intraoperatively, and nausea and vomiting after surgery. If used in day surgery the time to discharge is usually several hours after initial preparation. In spite of tumescent anesthesia's improved hemostasis, reduced hematoma, hyperpigmentation, and pre-dissection of the vein from surrounding tissue, its success depends very largely on surgeon expertise and precision of the injections. Also, when large volumes of solution are injected, the elimination of these fluids become an issue and may be at times inconvenient. This is now also thought to be major contributor to patient bruising, discomfort, except with highly proficient and experienced practitioners. Some studies suggest that in some cases, this technique may cause damage to laser fiber. Another inconvenient attributed to tumescent local anesthesia is that, as large amounts of liquid are injected, real time ultrasound imaging is rendered difficult, which is important especially to assess the vein closure process.

The possibility of interrupting pain pathways at multiple anatomic levels and the ability to provide excellent operating conditions without sedation or obtundation makes specific peripheral nerve blocks ideally suited for surgery of the lower extremity. Low incidence of perioperative complications, excellent postoperative analgesia and increased operating room efficiency, have contributed to a recent resurgence of interest in these techniques. The introduction of more effective, safer, long acting local anesthetics, as well as better equipment have further broadened the spectrum of applications of peripheral nerve blocks. Peripheral nerve blocks are ideally suited for lower extremity ambulatory surgery because of the peripheral location of the surgical site and the potential to block pain pathways at multiple levels. In contrast to other anesthetic techniques, such as general or spinal anesthesia, properly conducted peripheral nerve blocks avoid hemodynamic instability and pulmonary complications and allow real time ultrasound monitoring of treatment to determine the amount of energy delivered. This avoids inadequate treatments and facilitates post-operative pain management and timely discharge. Additional advantages of peripheral nerve blocks are that they are generally not contraindicated in patients taking anti-coagulants, they can be used in patients having lumbo-sacral disease and avoid the need for airway instrumentation.

When nerve block techniques are used along with minimally invasive laser procedures, vein treatment can be accomplished with superb results, regarding efficient vein closure, safety, pain, patient comfort and simplicity.

The method disclosed herein, preferably uses vascular laser procedures, associated with peripheral nerve block, taking advantage of their principal benefits, thus overcoming previously stated prior art's main drawbacks.

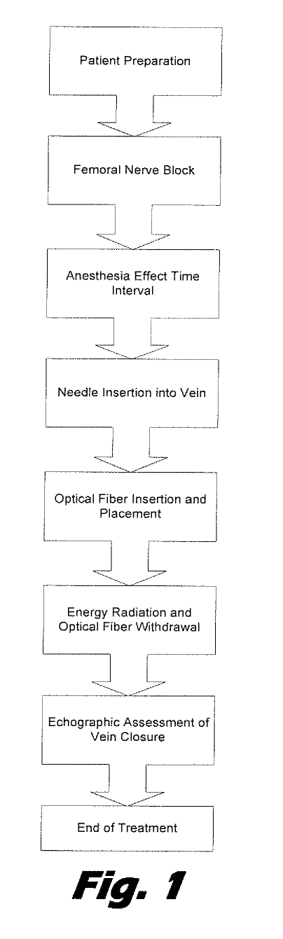

In a preferred embodiment, depicted in FIG. 1, the procedure consists in performing a femoral nerve block, in order to achieve a sensorial block but not a motor block, by means of a local, non tumescent anesthetic. A femoral nerve block carries low risk of complications and when combined with block of the sciatic nerve provides anesthesia of almost the entire lower extremity. If small saphenous vein (SSV) is treated, then the lower branch of the nerve may also be required to be blocked.

To accomplish femoral nerve block, an appropriate amount of local anesthetic is injected in the vicinity of femoral nerve close to the femoral artery under ultrasound control. After anesthesia takes effect, a needle is inserted through the skin into the vein to be treated and an optical fiber is introduced inside it. A typical procedure to carry out this task is to insert a guide wire inside the vein through needle's lumen, after the anesthesia has been introduced and takes effect. Once the guide wire is inserted, a dilator is used to slightly increase the size of the punction made onto the skin, in order to accomplish an easy catheter insertion. After catheter is inserted, guide wire is extracted and optical fiber is inserted through the catheter inside the vein and is advanced to the desired start position.

In a preferred embodiment, 20 ml of 0.5% lidocaine are used to accomplish the sensorial femoral block, injected under echographic, often called ultrasound, control. It has been found that the use of non-tumescent local anesthesia generally reduces the pain during and immediately after the treatment, contrary to prior belief, where it was always thought that a heat sink material around the vein was necessary to insulate nerves from local energy output, be it laser or RF in nature.

Some newly designed optical fibers (for instance, radial emitting fibers, as described in application Ser. Nos. 12/499,334, 12/395,455 and their subsequent applications), due to their blunt tip and flexibility, allow carrying out the fiber insertion procedure without needing a guide wire. As a consequence, after inserting the needle, an optical fiber is directly introduced through it (or a catheter) inside the vein, thus speeding up the whole vein treatment procedure. These type of fibers also provide enhanced uniformity in irradiation and more efficient treatment of vein walls. Their use in the present method is included by reference.

Once an optical fiber is placed in the appropriate position, the laser source connected to the fiber proximal end is activated and radiation is emitted at the fiber distal end while withdrawing the optical fiber, choosing appropriate irradiation parameters for each case. As a consequence, insufficient vein closure is achieved and the procedure is completed.

A main objective of this femoral block is to achieve sensory nerve function for blockage only, thus the least amount of lidocaine used yields the least probability for motor function to be affected. However, if lidocaine concentration is lowered too significantly, then it is possible for its anesthetic effect to be lost and the patient could feel pain. As a consequence, there is some minimal amount needed, which might differ by patient and procedure for an adequate and complete sensory block. It has been found in another preferred embodiment, that at least as low as a 0.1% lidocaine functioned well, while enhancing the likelihood of only blocking sensory nerves, avoiding motor impairing.

In other experiments, 20 ml of lidocaine with a concentration ranging from 0.1% to 0.5% were used to accomplish the femoral block, injected under echographic control. It was found, however, that 20 ml of 1% lidocaine also blocked motor function. Lidocaine solutions much above 0.5% are thus not preferred, as the likelihood of concomitant loss of motor control along with the desired sensory blockage becomes too great, rendering patient recovery period longer. Concentration of other local anesthetics, with differing activity, would have a somewhat different effective range of preferred use.

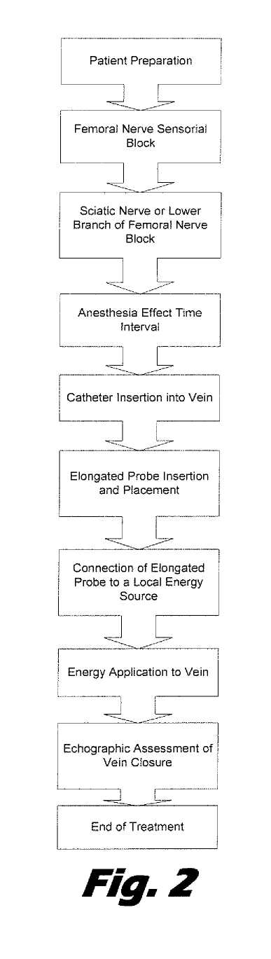

In another preferred embodiment, shown in FIG. 2, femoral nerve desensitization is combined with minimally invasive vein treatment using a local energy source. In order to perform anesthesia, different variations of the embodiment described in FIG. 1 can be used, depending on the specific treatment, patient features and physician experience and preferences. In other words, a femoral nerve block combined with block of the sciatic nerve or alternatively, a femoral nerve block below the main nerve with a shot at the lower branch of the nerve, near the knee, can be performed. Disregarding the method chosen, ultrasound guidance is used to make the anesthetic punction. After anesthesia takes effect, the technique continues by inserting an elongated probe, through a catheter, inside the vein to be treated. This elongated probe, which is inserted inside the vein to be treated, conveys energy from a local source to target tissue. Local energy sources may include, but not limited thereto, laser, RF, and thermal. Once elongated probe is placed in the appropriate position, it is connected to the local energy source, which is activated and energy is delivered at the probe distal end. Elongated probe can be withdrawn during treatment at a determined speed in order to cover a necessary vein length for achieving its closure. However, in case of special probe configurations in which energy is effectively delivered simultaneously all along an appropriate portion of the vein at a time, withdrawal is not required. As a consequence, closure of insufficient vein is achieved and the procedure is completed. In a preferred embodiment, the selected optical fiber is a radial emitting fiber and the selected laser wavelength is 1470±40 nm. Using radial emitting fibers allows for faster optical fiber insertion, minimizing perforation risks as well as pain during and after procedure due to even and uniform radiation. Since the 1470 nm wavelength is highly absorbed in water, the main component of living tissues, low energy densities can be used to efficiently, safely and reliably perform the vein treatment. One to five watt laser power levels delivered at 10-30 J/cm to the veins are now commonly producing excellent result with permanent closures of a wide range of varicose veins.

In addition to mentioned preferred embodiments, the method disclosed in the present invention can be performed by means of different optical fiber configurations, for example, bare fibers, side fibers, etc. and utilizing different wavelengths, for instance, 810 nm, 940 nm, 980 nm, 1320 nm, 1500 nm, 1940 nm, etc. The present inventions approach to anesthetic use works well to produce good closures with minimal or no pain to patients both during treatment and in post-operative phases.

Despite disclosed embodiments' reference to laser sources, the present invention can combine the described local, non tumescent anesthetic treatment with a variety of local energy emitting sources in order to perform vascular treatment, including thermal or radio frequency sources distributed at the distal end of an appropriate probe.

Femoral Block Anesthesia under Ultrasonic control is an excellent option to use in outpatients submitted to endovenous thermal ablations of the GSV above knee. It is easy to do, inexpensive, with a short learning curve and promotes a good anesthesia with none or minimal motor blockage.

The present invention is further illustrated by the following examples, but is not limited thereby.

### Example 1

A safe and effective femoral block procedure is achieved by sequentially carrying out the following steps:
1. Attach monitors and if necessary give light sedation.
2. Put patient in supine position with the operative leg slightly abducted.
3. Prepare the groin.
4. Place a transducer with the appropriate frequency range (10-12 MHz) along the inguinal crease. If the femoral artery and nerve are deep (>4 cm), use a 7 MHz transducer.
5. Optimize machine imaging capability; select appropriate depth of e d (usually within 1-3 cm), focus range and gain.
6. Identify the femoral artery. If the image shows more than 1 artery, scan more proximally (cephalad) to visualize the artery before the profunda femoris artery branches off.
7. Identify the femoral artery (FA). If the image shows more than 1 artery, scan more proximally (cephalad) to visualize the artery before the profunda femoris artery branches off.
8. Locate the femoral nerve externally to the FA within a triangular hyperechoic region, lateral to the FA and superficial to the iliopsoas muscle.
9. Place a skin wheal of lidocaine over the target.
10. Under ultrasound control, insert a needle through the skin wheal perpendicular to the transducer and the ultrasound beam and progress to the target.
11. Once satisfied with needle placement, inject lidocaine around the nerve and under Ultrasound guidance.
12. Scan proximally and distally to assess the extent of local anesthetic spread.

### Example 2

According to the first disclosed embodiment, 20 ml of 0.5% lidocaine with no vasoconstrictor were used to perform femoral nerve block. This local anesthetic is injected in the femoral nerve vicinity.

After anesthetic effect takes place, a radial emitting optical fiber is introduced, under ultrasound control, inside the great saphenous vein and advanced to the desired start position. Once in position, the optical fiber is connected to a 1470 nm diode laser source, which is activated and set at 6 W, in continuous mode. Then, laser radiation is emitted from the optical fiber while withdrawing it, leading to vein's closure. In order to accomplish an efficient closure of 10-12 diameter veins, 40-45 J/cm are applied to them.

Having described preferred embodiments of the invention with reference to the accompanying drawings, it is to be understood that the invention is not limited to the precise embodiments, and that various changes and modifications may be effected therein by skilled in the art without departing from the scope or spirit of the invention as defined in the appended claims.

What is claimed is:

1. A method for laser treatment of vascular abnormalities, such as venous insufficiency, employing a local, non-tumescent, anesthetic to desensitize a treatment area of a patient's lower extremity, prior to a laser treatment of said vascular abnormalities.

2. The method for laser treatment according to claim 1, wherein following introducing of said local anesthetic; introducing an optical fiber into a vein, requiring treatment, and intravenously applying said laser treatment thereafter.

3. The method according to claim 1, wherein employing said local anesthetic is done by the step of applying it to said lower extremity in the vicinity of a femoral nerve, and then applying it again in the vicinity of a sciatic nerve.

4. The method according to claim 3, wherein employing said local anesthetic is done by the further step of applying said local anesthetic also in the vicinity of a sciatic nerve.

5. The method according to claim 3, wherein employing said local anesthetic is done by the further step of applying said local anesthetic also in the vicinity of said femoral nerve's lower branch.

6. A method for laser vascular treatment, comprising the steps of:
applying a local anesthetic to a lower extremity in the vicinity of area to be treated;
desensitizing a treatment area of said patient's lower extremity prior to beginning treatment and maintaining a patient's motor control unimpaired; and
applying laser radiation and performing said laser treatment.

7. The method for laser treatment according to claim 6, further comprising, prior to applying said local anesthetic, selecting said local anesthetic as a lidocaine solution, having a concentration of no greater than about 0.5%

8. The method according to claim 6, wherein said step of applying local anesthetic to said lower extremity is restricted to applying it in the vicinity of a femoral nerve.

9. The method according to claim 8 wherein said step of applying said local anesthetic involves applying it again in the vicinity of a sciatic nerve.

10. The method according to claim 8, wherein said step of applying said local anesthetic involves applying it again in the vicinity of said femoral nerve's lower branch.

11. The method according to claim 7, wherein said step of selecting said lidocaine solution is restricted to selecting a concentration of between about 0.1% and about 0.5% of said lidocaine.

12. A method for local energy treatment of vascular abnormalities, such as venous insufficiency, employing a local, non-tumescent, anesthetic to desensitize a treatment area of a patient's lower extremity prior to a local energy treatment of said vascular abnormalities.

13. A method for local energy vascular treatment, maintaining unimpaired motor control of a patient by applying of a local anesthetic to desensitize a treatment area of said patient's lower extremity prior to a local energy treatment.
